(19) United States
(12) Patent Application Publication
(10) Pub. No.: US 2026/0083871 A1
(43) Pub. Date: Mar. 26, 2026

(54) STERILIZATION METHODS

(71) Applicant: Noxilizer, Inc., Hanover, MD (US)

(72) Inventor: David Opie, Hanover, MD (US)

(21) Appl. No.: 19/338,538

(22) Filed: Sep. 24, 2025

Related U.S. Application Data
(60) Provisional application No. 63/698,288, filed on Sep. 24, 2024.

(51) Int. Cl. A61L 2/24; A61L 2/08; A61L 2/20; A61L 101/02; A61L 101/44

(52) U.S. Cl. CPC A61L 2/24; A61L 2/087; A61L 2/20; A61L 2/206; A61L 2101/02; A61L 2101/44; A61L 2103/15; A61L 2202/121; A61L 2202/122; A61L 2202/14

(57) ABSTRACT

Methods and systems for sterilizing medical devices. An illustrative method of sterilizing a medical device may comprise placing the medical device in a sterilization chamber, locking a door of the sterilization chamber, and performing one or more steps of a sterilization procedure. The one or more steps of the sterilization procedure may comprise exposing the medical device to a sterilant and monitoring a concentration of the sterilant and/or a by-product of the sterilization procedure. After the sterilization procedure is complete the door of the sterilization chamber may be unlocked when the concentration of the sterilant and/or the by-product of the sterilization procedure is below a predetermined threshold.

### CROSS REFERENCE TO RELATED APPLICATIONS

The present application claims the benefit of and priority to U.S. Provisional Patent Application 63/698,288, filed Sep. 24, 2024, titled STERILIZATION METHODS, the disclosure of which is incorporated herein by reference.

### TECHNOLOGY FIELD

The present application relates generally to a sterilization method, and in particular, to sterilization methods with improved safety measures.

### BACKGROUND

A wide variety of medical devices have been developed for medical use. Some devices are fully implantable (for example, orthopedic implants, stents and various electrical stimulators) to replace or repair bone, in the bloodstream or elsewhere in the body. Other devices are introduced and removed from the body in a single procedure (for example, endoscopes, catheters, and guidewires). Still other devices are used for introducing materials to or extracting material from the body (for example, syringes). Still other products are used in repairing or otherwise treating the body (for example, sutures and various staples).

There are many different sterilization methods and/or systems that may be used to sterilize devices. Steam sterilization (hereinafter, simply referred to as “AC”) and ethylene oxide gas sterilization (hereinafter, simply referred to as “ETO sterilization”) have been widely used as sterilizing methods of medical instruments. Gamma radiation may also be used for a range of devices. Additionally, hydrogen peroxide and nitrogen dioxide have been used for sterilization

What may be desirable are sterilization methods and systems that detect levels of sterilant and by-products of the sterilization process.

### SUMMARY

This disclosure provides methods of sterilizing devices, such as, but not limited to medical devices. This summary is not intended to describe each disclosed embodiment or every implementation of the invention.

In a first example, an illustrative of sterilizing a medical device may comprise placing the medical device in a sterilization chamber, locking a door of the sterilization chamber, and performing one or more steps of a sterilization procedure. The one or more steps may comprise exposing the medical device to a sterilant and monitoring a concentration of the sterilant and/or a by-product of the sterilization procedure. After the sterilization procedure is complete, the method may further comprise unlocking the door of the sterilization chamber in response to fulfilling both a) the monitored concentration of the sterilant and/or the by-product of the sterilization procedure being below a predetermined threshold and b) a request to unlock the chamber is received.

Alternatively or additionally to any of the examples above, in another example, the sterilant may be nitrogen dioxide.

Alternatively or additionally to any of the examples above, in another example, the predetermined threshold may be 10 parts per million of nitrogen dioxide.

Alternatively or additionally to any of the examples above, in another example, the predetermined threshold may be 1 part per million of nitrogen dioxide.

Alternatively or additionally to any of the examples above, in another example, the by-product may be nitrogen oxide.

Alternatively or additionally to any of the examples above, in another example, the predetermined threshold may be a change in slope of a plot of a log of the concentration of nitrogen oxide versus time.

Alternatively or additionally to any of the examples above, in another example, the sterilant may be ethylene oxide.

Alternatively or additionally to any of the examples above, in another example, the predetermined threshold may be 18 parts per billion of ethylene oxide.

Alternatively or additionally to any of the examples above, in another example, the predetermined threshold may be 13 parts per billion of ethylene oxide.

Alternatively or additionally to any of the examples above, in another example, the sterilant may be radiation.

Alternatively or additionally to any of the examples above, in another example, the by-product may be ozone.

Alternatively or additionally to any of the examples above, in another example, the predetermined threshold may be 0.10 parts per million of ozone.

In another example, a method of sterilizing a medical device may comprise placing the medical device in a sterilization chamber, receiving at an access control system a command to lock a door of a sterilization system, locking a door of the sterilization system, and performing one or more steps of a sterilization procedure. The one or more steps of the sterilization procedure may comprise exposing the medical device to a sterilant and monitoring a concentration of the sterilant and/or a by-product of the sterilization procedure. In response to the concentration of the sterilant and/or the by-product of the sterilization procedure is below a predetermined threshold, the method may further comprise enabling access to the sterilization chamber, receiving at an access control system a request to unlock a door of the sterilization chamber, and either if access has been enabled, unlocking the door or if access has not been enabled, denying the request to unlock the door.

In another example, a sterilization apparatus may comprise a sterilization chamber having a lockable door, at least one chemical sensor positioned within a chamber of the sterilization chamber, an access control system in electronic communication with the lockable door, a control and monitoring system in electronic communication with the at least one chemical sensor and the access control system, and a sterilant source in fluid communication with the chamber of the sterilization chamber, the sterilant source providing a sterilant to the chamber of the sterilization chamber. The access control system may be configured to lock the lockable door at a start of a sterilization procedure and unlock the lockable door as follows: monitoring a concentration of the sterilant or a by-product of the sterilization procedure after the sterilant has been introduced to the chamber, determining the concentration of the sterilant or by-product is below a predetermined threshold and, in response, enabling access to the chamber, receiving a request to unlock a door of the sterilization chamber, and either: if access has been enabled, unlocking the door, or if access has not been enabled, denying the request to unlock the door.

Alternatively or additionally to any of the examples above, in another example, the sterilization apparatus may further comprise an enclosure surrounding the sterilization chamber.

Alternatively or additionally to any of the examples above, in another example, the sterilization apparatus may further comprise at least one chemical sensor positioned within the enclosure.

Alternatively or additionally to any of the examples above, in another example, the sterilization apparatus may further comprise one or more leak sensors external to the sterilization chamber.

Alternatively or additionally to any of the examples above, in another example, the sterilant may be nitrogen dioxide.

Alternatively or additionally to any of the examples above, in another example, the sterilant may be ethylene oxide.

Alternatively or additionally to any of the examples above, in another example, the sterilant may be an electron beam.

### BRIEF DESCRIPTION OF THE DRAWINGS

The invention may be more completely understood in consideration of the following detailed description of various embodiments in connection with the accompanying drawings, in which:

FIG. 1 is an illustrative sterilization apparatus in block form;

FIG. 2 is a flow chart of an illustrative sterilization process;

FIG. 3 is an illustrative flow chart of an illustrative method for performing a sterilization process using nitrogen dioxide;

FIG. 4 is an illustrative flow chart of an illustrative method for performing a sterilization process using ethylene oxide;

FIG. 5 is an illustrative flow chart of an illustrative method for performing a sterilization process using chlorine dioxide; and

FIG. 6 is an illustrative flow chart of an illustrative method for performing a sterilization process using electron beam radiation.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION OF EXEMPLARY EMBODIMENTS

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure. The recitation of numerical ranges by endpoints includes all numbers within that range (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

In some current sterilization procedures, once the sterilization is complete, the system may be configured to perform a predetermined number of evacuation and refill cycles. After the predetermined number of evacuation and refill cycles are completed (sometimes referred to as “rinsing”), the sterilization chamber is opened, such as by opening an access door. Sterilized product is then removed, and left to outgas in a ventilated room. In general operator safety is not closely controlled using environmental controls and, instead, relies on the use of parametric process values developed during sterilization cycle development and the use of respirators and personal safety equipment.

However, safety standards are continuously evolving. For example, in a recent final rule from the Environmental Protection Agency (EPA), an amount of ethylene oxide (ETO) that exits a building has been subject to regulation. Further, pursuant to new rules and regulations, within a building, the use of respirators will be required when ETO gas is above a predetermined level set in the range of parts per billion (ppb). It is contemplated that respirators may be required for ETO gas concentrations above a relatively low threshold, such as 13 ppb, applicable to the environment outside the sterilization chamber. The lower limit may be based on detectable concentration levels; that is, available technology may not be capable of reliably detecting ETO concentrations below the set threshold. Given the proposed link between technical capability and the threshold, it may well be that improved technology may facilitate still lower safety threshold setting. The present disclosure is directed towards sterilization methods and systems which improve operator safety by limiting operator exposure to a sterilant or chemical by-products of the sterilization process.

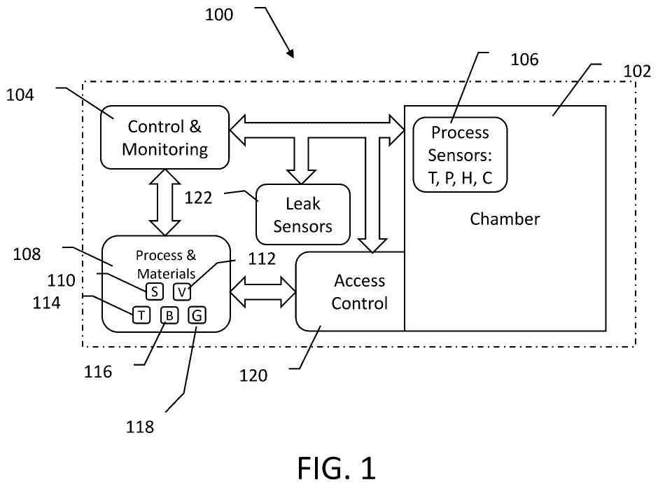

FIG. 1 shows an illustrative, non-limiting, sterilization apparatus 100 in block form. The sterilization apparatus 100 may take a range of sizes, from industrial versions that take up entire rooms at a plant, with volumes in the thousands of liters to portable versions that may be used in the healthcare setting such as a hospital, with volumes in the tens to hundreds of liters. The sterilization apparatus 100 includes a sterilization chamber 102. The sterilization chamber 102 may typically be a chamber having a door that can be sealed for airtightness.

The sterilization apparatus 100 may further include a control and monitoring system 104. The control and monitoring system 104 may include a microprocessor(s), microcontroller(s), user interface, memory, and/or various sensing, measuring, conditioning, and communication circuits, as needed. For example, the control and monitoring system 104 may include a graphical user interface (a screen, whether touch or not), a keyboard/mouse/touchscreen or other data/command entry station, a processor such as a microprocessor or microcontroller having associated memory for performing methods as disclosed herein and others known in the art. The control and monitoring system 104 may take a range of different forms.

The control and monitoring system 104 may be communicatively coupled (such as by wire, optical, or wireless connection) to the sterilization chamber 102 to obtain various data from sensors 106 therein. Some illustrative sensors 106 may include, but are not limited to, temperature, pressure, humidity, chemical, and/or other sensors. The specific sensors may vary with the particular sterilization chemistry or process being used. For example, one or more chemical sensors may be present based on the sterilant and/or chemical by-products produced during the sterilization procedure. Redundant sensors may be provided in a sterilization chamber 102, although this is not required.

The sterilization process is controlled by the control and monitoring system 104 using the process and materials technology 108 that is available. The process and materials may be based, at least in part, on the type of sterilant used. The process and materials technology 108 may include, for example, a sterilant source 110 (sometimes a pressurized canister holding a sterilant but not necessarily), a vacuum source 112, a temperature control apparatus 114, a blower 116, and/or other gas sources 118. The sterilant 110 may be any suitable sterilant, including, but not limited to steam, ethylene oxide, nitrogen dioxide, hydrogen peroxide, chlorine dioxide, vaporized peracetic acid, for example. Cobalt-60 may be used in sterilization systems using gamma irradiation. A vacuum 112 may be applied using any suitable vacuum pump design (liquid ring, rotary vane, running claw, etc.). The temperature control apparatus 114 may include, in various forms, a resistive heating apparatus, a Peltier cooling apparatus, a refrigeration system, and/or the use of piping in or on the walls of the chamber 102 through which heated or cooled fluid is pumped. A blower 116 can be provided to circulate the gasses within the chamber, ensuring mixing of gasses and preventing localized concentrations and condensation. Additional gas sources 118 can be provided as well, including non-sterilant gas sources such as sources of water vapor and/or dry air, or other particular gasses (nitrogen, oxygen, and/or inert gasses, as desired).

The sterilization apparatus 100 may further include an access control system 120. The access control system 120 may be configured to prevent a user from opening or otherwise accessing the chamber 102 during a sterilization procedure. It is further contemplated that the access control system 120 may prevent a user from opening or otherwise accessing the chamber 102 after a sterilization procedure is complete if the chemical concentrations of the sterilant and/or chemical by-products of the sterilization procedure within the chamber 102 are not below a predetermined concentration. For example, the control and monitoring system 104 may be in communication (e.g., wired, wireless, and/or optical communication) with the chemical sensors 106 and the access control system 120. In some examples, the control and monitoring system 104 may not issue a command to unlock or release the access control system 120 until the concentration of the sterilant and/or chemical by-products of the sterilization procedure are below a predetermined concentration.

In some embodiments, the sterilization apparatus 100 may include one or more leak sensors 122. The one or more leak sensors 122 may be exterior to the chamber 102 to determine if gases are leaking from an interior of the chamber 102 and into the room with the sterilization apparatus 100. The leak sensors 122 may be chemical sensors configured to detect concentrations of the sterilant and/or chemical by-products of the sterilization procedure. In other examples, the one or more leak sensors 122 may be configured to detect a flow of air/gas from the chamber 102. The one or more leak sensors 122 may be in communication (e.g., wired, wireless, and/or optical communication) with the control and monitoring system 104. If a flow of gas is detected from the chamber 102 or if a chemical concentration within the room housing the sterilization apparatus 100 exceeds a predetermined concentration, the control and monitoring system 104 may be configured to stop the sterilization procedure, issue an alert (e.g., activate a warning light, activate an audio alert, transmit an alert to a remote device, or the like), prevent a user from entering the room (e.g., by locking an access door), start or increase external ventilation, and/or combinations thereof.

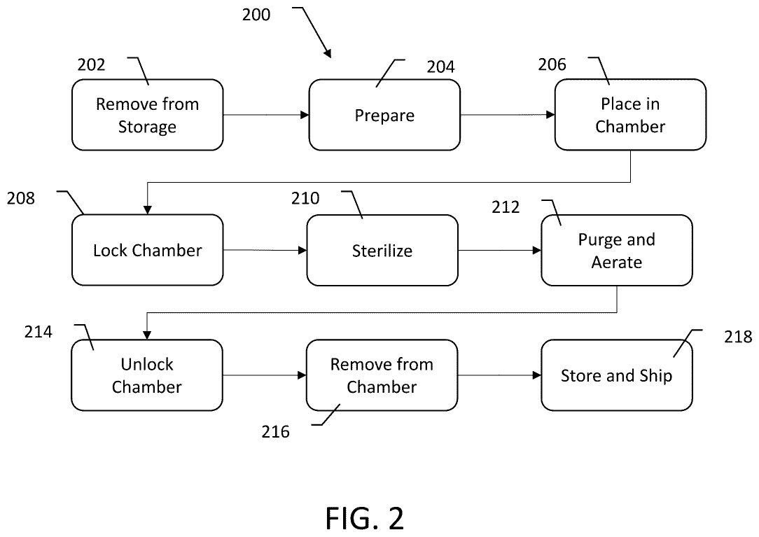

A prior art sterilization process 200 is shown in block form in FIG. 2. The sterilization process or method 200 starts with removal of the product to be sterilized from storage, as shown at block 202. In some examples, the storage location may be a refrigerated location, as various drugs, biologics, and devices can be stored at cool temperatures to, for example, prevent degradation thereof. The product may be prepared for sterilization, as shown at block 204. It is contemplated that the specific preparation steps may vary based on the type of product to be sterilized and/or the type of sterilant. Next, the product may be placed in the chamber, as shown at block 206. The sterilization chamber may then be closed and locked, as shown at block 208. It is contemplated that the sterilization chamber may be manually locked by a user or may be automatically locked with a control program.

Once the sterilization chamber is locked, the sterilization process may be performed, as shown at block 210. In many instances, the sterilization process 210 begins with a series of steps that pre-condition the product sample and/or sterilization chamber. For example, the chamber and product may be dried or humidified (or dried then humidified). The sterilization process may use the specific steps a particular sterilant requires. Next the chamber is purged and the product is aerated, as shown at block 212. Sometimes the aeration process can extend for hours. Once aeration is complete, the sterilization chamber may be unlocked (e.g., manually or automatically via a control program and electronic communication), as shown at block 214. The product is then removed from the chamber, as shown at block 216. In some instances, aeration occurs both before and after removal from the chamber. For example, the product may be removed from the sterilization chamber and left in a room (such as, but not limited to, the sterilization room) for further aeration. The product is then returned to storage/refrigeration and/or shipped to another location, as shown at block 218.

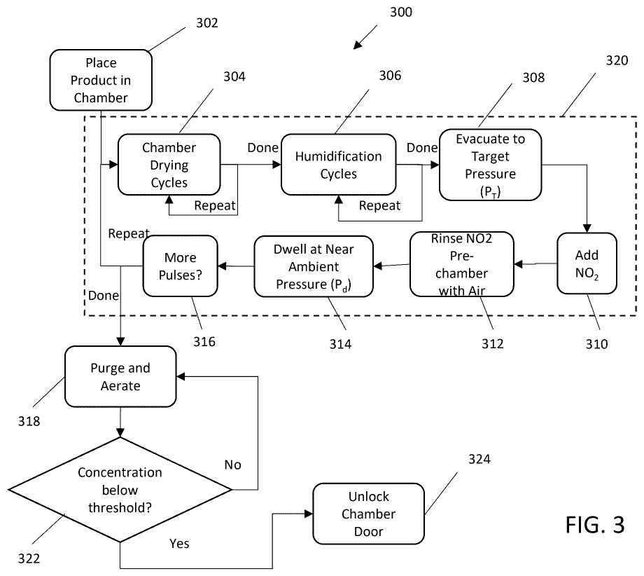

As described herein, it may be desirable to monitor the sterilant concentration and/or the chemical by-products within the sterilization chamber during and after the sterilization process. FIG. 3 is an illustrative flow chart of an illustrative method 300 for performing a sterilization process using the sterilizing apparatus 100 and nitrogen dioxide (NO2). Additional methods and systems for performing sterilization using NO2 are described in commonly assigned U.S. Patent Publication Number 2023/0364286, titled STERILIZATION METHOD, the disclosure of which is hereby incorporated by reference.

To begin, an object to be sterilized may be placed in the sterilization cavity of the sterilizing chamber 102, as shown at block 302. In one example, the object may be a filled syringe. The filled syringe may include contents within the barrel thereof. For example, the contents may include a therapeutic agent. However, this is not required. Other objects may include, but are not limited to, orthopedic implants, stents, various electrical stimulators, endoscopes, catheters, guidewires, sutures, various staples, or the like.

Optionally, the sterilization procedure may be performed such that a temperature of the contents of the syringe does not vary by more than ±5° C. or by more than ±3° C. In other examples, the sterilization process may be performed without concern for temperature changes of the object to be sterilized, or with wider allowed variation, or with actually changing the temperature of the object such as by intentionally cycling pressure and/or temperature to warm or cool product. In some cases, the temperature of the contents may be maintained within a temperature range of about 15-25° C.

Once the object has been placed in the sterilization cavity, the door of the sterilizing chamber 102 may be closed and locked. The door may be manually locked by a user or may be automatically locked using, for example, the access control system 120. For example, the access control system 120 may include an electronically actuated locking system which locks the door of the sterilizing chamber 102 in response to a command received from the control and monitoring system 104. The command may be issued in response to the closing of the door and/or the initiation of the sterilization procedure. The sterilization cavity is then dried using a series of evacuation and fill steps, as shown at block 304. Drying the sterilization cavity may provide a controlled starting place, or controlled process conditions for each “pulse” of the sterilization process.

With the sterilization cavity now in a known state, the sterilization cavity is humidified in a controlled manner, with a series of evacuation and fill steps, until a target relative humidity is reached, as shown at block 306. The target relative humidity may be, for example and without limitation, in the range of about 25% to about 90%, or about 40% to about 80%, or about 80%. Target relative humidity may be influenced by process factors including surface temperatures and pressures to be used, where the target relative humidity may be, in some examples, selected to prevent condensation during all phases of the sterilization procedure. In some cases, the relative humidity may be monitored using infrared (IR) or visible light detectors.

Next, the chamber is depressurized to a target pressure, as shown at block 308. Target pressures P_{T} may be in the range of, for example, 200 Torr to 500 Torr when performing the method with a pre-filled syringe. In some cases, P_{T} may about in the range of about 450 Torr. In other examples, P_{T} may be higher or lower, if desired. When humidity is added prior to adding NO2, the humidification may be accomplished by recirculating gas from the sterilization chamber through a humidifying element. If preceding steps are performed to leave the dried chamber in a depressurized state, the additional depressurization step at 308 may optionally be omitted.

Next, a quantity of NO2 is introduced into the sterilization cavity, as shown at block 310. NO2 may be introduced from a gas supply system with the use of a separate, smaller pre-chamber or buffer tank. In some examples, the sterilization cavity may have a volume in the range of about 20 L to 5000 L, with a corresponding pre-chamber buffer tank having a volume of in the range of about 4 L to 60 L. Other volumes may be used, as desired.

A concentrated mass of NO2 is placed in the pre-chamber or buffer tank at a lower pressure than the target pressure P_{T} of the sterilization cavity, where the lower pressure in the pre-chamber or buffer tank is selected to maintain the NO2 in the buffer tank in its gaseous state and prevent condensation. Next, the pre-chamber or buffer tank may be pressurized by adding ambient or dry air until a pressure in the pre-chamber or buffer tank is reached that is higher than the pressure in the sterilization cavity. A valve is then opened to release the NO2 and air mix in the pre-chamber or buffer tank into the sterilization cavity. The concentration of the NO2 may be measured using IR detectors or visible light detectors. Additional air is then rinsed through the pre-chamber or buffer tank and into the sterilization cavity, as shown at block 312. The volume of added air at the resident pressure may be approximately six times the volume of the pre-chamber or buffer tank, thus ensuring mixing of the NO2 and full introduction into the sterilization cavity.

Steps 310 and 312 add a quantity of air and NO2 to the sterilization cavity, leaving the sterilization cavity with dwell pressure, P_{d} which may be in the range of about 600 Torr, for example, in the range of 500 Torr to ambient pressure, or even above ambient pressure for example up to about ambient pressure plus 100 Torr. In some examples, the dwell pressure may be in the range of about 550 to about 650 Torr. Dwell pressure may also be understood relative to ambient pressure, being in the range of about 200 Torr below ambient pressure to about 100 Torr above ambient pressure, or in the range of about 100 Torr below ambient pressure to ambient pressure. In some examples, the dwell pressure exceeds the target pressure by at least about 150 Torr. In some examples, the dwell pressure is at ambient pressure, which may be an average ambient pressure for the location, or may be determined by sensing ambient pressure using an external pressure sensor. The quantity of NO2 introduced in step 310 may be sufficient to result in an NO2 concentration in the sterilization cavity in the range of about 2-20 milligrams per liter (mg/L), or higher or lower if desired. However, the concentration of NO2 in the buffer tank may be several times higher than the resulting concentration of NO2 in the sterilization cavity. For example, assuming the sterilization chamber 102 has a volume of 4000 L, and the buffer tank has a volume of 40 L, the concentration of NO2 in the buffer tank may be in the range of 200 mg/L to attain 2 mg/L concentration in the sterilization cavity, or 2000 mg/L to attain 20 mg/L concentration in the sterilization cavity. Other values may be used. Specific process parameters may vary depending on the device or object to be sterilized, including features such as surface contours, materials, material compatibility, whether moving parts are present that may be subject to movement due to pressure changes, the capability of materials, including those of the sterilized object and any material (medicine, biologic, etc.) contained in the sterilized object, to withstand heat, pressure, humidity, and NO2 itself.

The sterilization cavity may be held at stable conditions for a predetermined dwell time, as shown at block 314. For example, the humidity, NO2 concentration, and air in the sterilization cavity may remain fixed for a period of time in the range of about 4 minutes to 15 minutes. However, dwell times less than 4 minutes or greater than 15 minutes may be used, as desired. It should be noted that after the vacuum is applied at step 308, each of steps 310 and 312 raise the pressure in the sterilization cavity from the target pressure P_{T} to the dwell pressure P_{d}. As a result, the target pressure of step 308 is not the same as the pressure in the sterilization cavity during the dwell step 314.

At the end of any given dwell step, the chamber is evacuated, and NO2 that is evacuated is scrubbed/removed from exhaust gas. The process 320 is repeated for a number of pulses, as indicated at block 316. The number of pulses may range between 2 to 8 pulses. However, in some cases there may be only one pulse or there may be more than 8 pulses, as desired. When an additional pulse is called for in the sterilization routine, the process returns to step 304 to again place the sterilization cavity in a known state. When the process returns to block 304, the evacuation and refilling steps that are performed will evacuate NO2 from the prior dwell (step 314) from the sterilization chamber, which in turn will require treating the exhaust gas that results to reduce the exhausted quantity of NO2 to an environmentally acceptable level.

Once the sterilization routine is complete (e.g., the process 320 has been repeated for a predetermined number of pulses), the sterilization cavity may be purged and aerated, as shown at block 318. The sterilization cavity may be aerated through a series of evacuations or rinsing air through the sterilization cavity. For example, a load of air may be pulled through the sterilization cavity. To purge the sterilization cavity, the gas mixture may be exhausted via an exhausting apparatus to scrub the NO2 and nitric acid before the gas mixture is released to the ambient environment. If desired, the purge and aeration process 318 may include modifying the temperature of the sterilized object such as by using heated or cooled air during purge steps, and by heating or cooling the sterilization chamber walls.

The purge and aeration process 318 may comprise rinsing the sterilization chamber with air following the dwell step while monitoring a residual gas in the chamber using a residual gas sensor until a residual gas concentration falls below a predetermined safety threshold, as shown at block 322. For example, an NO2 sensor in the chamber or in a flow path associated with the chamber, such as in an exhaust flow path, may be used to monitor NO2 levels during aeration until such levels fall below a threshold. The purge and aeration process 318 may be repeated until the NO2 concentration falls below a predetermined threshold. The predetermined threshold concentration may be based on known safe concentration of NO2 and/or detectable levels. In some embodiments, the predetermined threshold concentration may be based, at least in part, on an NO2 concentration that ensures that chemical by-products of the sterilization process are also at safe levels. It is contemplated that the predetermined threshold of NO2 may be 1 parts per million (ppm). However, the predetermined threshold may be less than 1 ppm or more than 1 ppm in some instances. For example, the predetermined threshold concentration may be those listed by safety regulators. In some examples, the predetermined threshold concentration may be higher than a concentration listed by regulators as a safe level. For example, there may be means adjacent to the sterilization chamber to mitigate sterilant in the sterilization chamber. For example, in some cases, dilution in the air that surrounds the sterilization chamber may be sufficient to reduce a residual sterilant level to a safe level. Alternatively, an air curtain or air exchange in the room surrounding the sterilization chamber may be sufficient to render the sterilant escaping the sterilization chamber safe.

It is contemplated that the NO2 sensor may be configured to detect NO2 concentrations in the range of about 0.1 ppm to about 10 ppm. However, the NO2 sensor may be configured to detect other concentrations, as desired. Once the NO2 concentration is below the predetermined threshold, the control and monitoring system 104 may issue a control command to the access control system 120 to unlock the door the sterilization chamber 102, as shown at block 324. The door may not be unlocked or open until the NO2 concentration is below the predetermined threshold. For example, the user may not bypass the access control system 120 to open the sterilization chamber 102 while NO2 concentrations are above the predetermined threshold.

In some examples, a by-product of the sterilization process may be monitored as a proxy for NO2 concentration. For example, NO may be measured. Once the concentration of NO drops below a predetermined threshold, the process is complete and NO2 concentrations are at a safe level. For example, the log of the concentration of NO versus time may be plotted. A change in the slope of the plot may be indicative of concentration levels of NO2 and other chemical by-products being at a safe level. The control and monitoring system 104 may analyze the slope of the plot and when the slope changes, the control and monitoring system 104 may issue a command to the access control system 120 to unlock the door to the sterilization chamber 102.

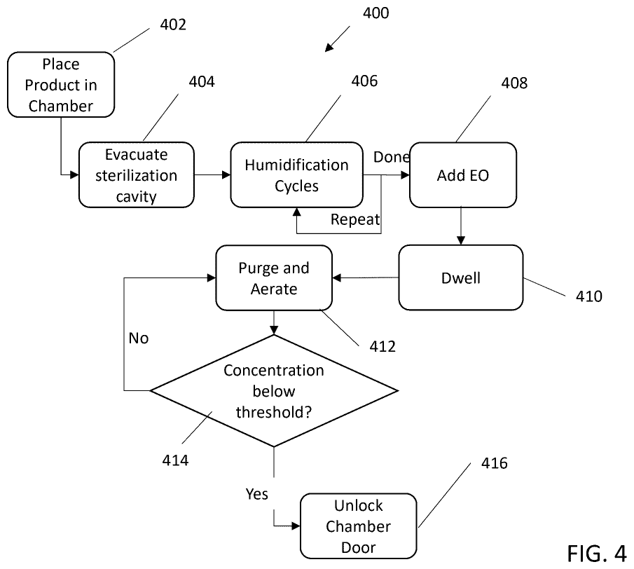

FIG. 4 is an illustrative flow chart of a method 400 for sterilizing an object using the sterilizing apparatus 100 and ethylene oxide (ETO). To begin, an object to be sterilized may be placed in the sterilization cavity of the sterilizing chamber 102, as shown at block 402. Once the object has been placed in the sterilization cavity, the door of the sterilizing chamber 102 may be closed and locked. The door may be manually locked by a user or may be automatically locked using, for example, the access control system 120. For example, the access control system 120 may include an electronically actuated locking system which locks the door of the sterilizing chamber 102 in response to a command received from the control and monitoring system 104. The command may be issued in response to the closing of the door and/or the initiation of the sterilization procedure. A vacuum may be applied to the sterilization cavity to lower the pressure of the cavity, as shown at block 404.

With the sterilization cavity now in a known state, the sterilization cavity is humidified in a controlled manner, until a target relative humidity is reached, as shown at block 406. The target relative humidity may be influenced by process factors including surface temperatures and pressures to be used, where the target relative humidity may be, in some examples, selected to prevent condensation during all phases of the sterilization procedure. In some cases, the relative humidity may be monitored using infrared (IR) or visible light detectors.

Next, a quantity of ETO is introduced into the sterilization cavity, as shown at block 408. In some cases, ETO may be mixed with another gas, such as, but not limited to dichlorodifluoromethane, to reduce the flammability of ETO. In other examples, 100% ETO may be used.

The sterilization cavity may be held at stable conditions for a predetermined dwell time, as shown at block 410. For example, the humidity, ETO concentration, and air in the sterilization cavity may remain fixed for a period of time in the range of about 2 hours to about 6 hours. However, dwell times less than 2 hours or greater than 6 hours may be used, as desired. In some cases, the dwell time may be based, at least in part, on a temperature of the sterilization cavity. It should be noted that after the vacuum is applied at step 404, each of steps 406 and 408 may raise the pressure in the sterilization cavity from an initial pressure after vacuum evacuation to a dwell pressure. However, this is not required.

Once the sterilization routine is complete, the sterilization cavity may be purged and aerated, as shown at block 412. The sterilization cavity may be aerated through a series of evacuations or rinsing air through the sterilization cavity. For example, a load of air may be pulled through the sterilization cavity. In some cases, the purge may be a vacuum purge. To purge the sterilization cavity, the gas mixture may be exhausted via an exhausting apparatus to scrub the ETO before the gas mixture is released to the ambient environment. If desired, the purge and aeration process 412 may include modifying the temperature of the sterilized object such as by using heated or cooled air during purge steps, and by heating or cooling the sterilization chamber walls. The purge and aeration process 412 may comprise rinsing the sterilization chamber with a non-sterilant containing gas, such as, but not limited to, air, nitrogen, or the like, following the dwell step while monitoring a residual gas in the chamber using a residual gas sensor until a residual gas concentration falls below a predetermined safety threshold, as shown at block 414. For example, an ETO sensor in the chamber or in a flow path associated with the chamber, such as in an exhaust flow path, may be used to monitor ETO levels during aeration until such levels fall below a threshold. The purge and aeration process 412 may be repeated until the ETO concentration falls below a predetermined threshold. It is contemplated that the purge and aeration process 412 may be repeated any number of times, such as, but not limited to, less than five times, more than five times, more than 10 times, more than 50 times, more than 100 times, etc. The number of times the purge and aeration process 412 is repeated may depend, at least in part, on the process and the depth of vacuum for each purge. The predetermined threshold concentration may be based on known safe concentration of ETO and/or detectable levels of ETO. In some cases, the predetermined threshold concentration of ETO may be 18 parts per billion (ppb) or less or 13 ppb or less. However, other concentrations are contemplated. For example, the predetermined threshold concentration may be those listed by safety regulators. In some examples, the predetermined threshold concentration may be higher than a concentration listed by regulators as a safe level. For example, there may be means adjacent to the sterilization chamber to mitigate sterilant in the sterilization chamber. For example, in some cases, dilution in the air that surrounds the sterilization chamber may be sufficient to reduce a residual sterilant level to a safe level. Alternatively, an air curtain or air exchange in the room surrounding the sterilization chamber may be sufficient to render the sterilant escaping the sterilization chamber safe.

Once the ETO concentration is below the predetermined threshold, the control and monitoring system 104 may issue a control command to the access control system 120 to unlock the door the sterilization chamber 102, as shown at block 416. The door may not be unlocked or open until the ETO concentration is below the predetermined threshold. For example, the user may not bypass the access control system 120 to open the sterilization chamber 102 while ETO concentrations are above the predetermined threshold.

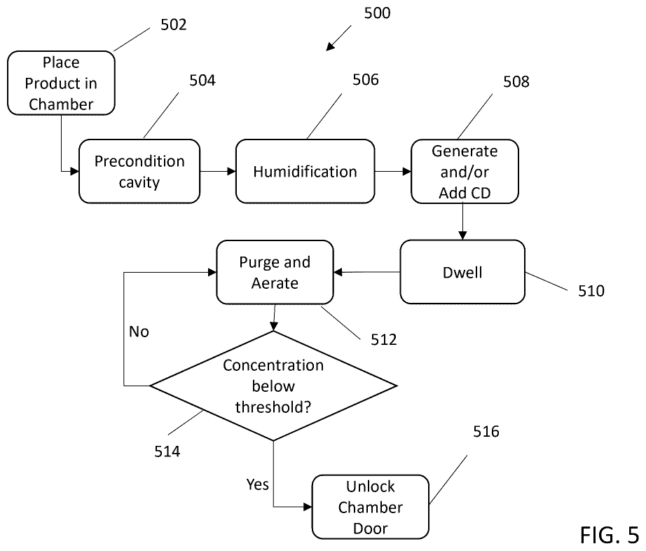

FIG. 5 is an illustrative flow chart of a method 500 for sterilizing an object using the sterilizing apparatus 100 and chlorine dioxide (CD). To begin, an object to be sterilized may be placed in the sterilization cavity of the sterilizing chamber 102, as shown at block 502. Once the object has been placed in the sterilization cavity, the door of the sterilizing chamber 102 may be closed and locked. The door may be manually locked by a user or may be automatically locked using, for example, the access control system 120. For example, the access control system 120 may include an electronically actuated locking system which locks the door of the sterilizing chamber 102 in response to a command received from the control and monitoring system 104. The command may be issued in response to the closing of the door and/or the initiation of the sterilization procedure. A vacuum may be applied to the sterilization cavity to lower the pressure of the cavity, as shown at block 504.

With the sterilization cavity now in a known state, the sterilization cavity is humidified in a controlled manner, until a target relative humidity is reached, as shown at block 506. The target relative humidity may be influenced by process factors including surface temperatures and pressures to be used, where the target relative humidity may be, in some examples, selected to prevent condensation during all phases of the sterilization procedure. In some cases, the relative humidity may be monitored using infrared (IR) or visible light detectors.

Next, a quantity of CD is introduced into the sterilization cavity, as shown at block 508. In some cases, CD may be generated on site. However, this is not required. The sterilization cavity may be held at stable conditions for a predetermined dwell time, as shown at block 510. For example, the humidity, CD concentration, and air in the sterilization cavity may remain fixed for a period of time in the range of about 30 minutes to about 60 minutes. However, dwell times less than 30 minutes or greater than 60 minutes hours may be used, as desired. In some cases, the dwell time may be based, at least in part, on a temperature of the sterilization cavity and/or a concentration of the CD. It should be noted that after the vacuum is applied at step 504, each of steps 506 and 508 may raise the pressure in the sterilization cavity from an initial pressure after vacuum evacuation to a dwell pressure. However, this is not required.

Once the sterilization routine is complete, the sterilization cavity may be purged and aerated, as shown at block 512. The sterilization cavity may be aerated through a series of evacuations or rinsing air through the sterilization cavity. For example, a load of air may be pulled through the sterilization cavity. In some cases, the purge may be a vacuum purge. To purge the sterilization cavity, the gas mixture may be exhausted via an exhausting apparatus to scrub the CD before the gas mixture is released to the ambient environment. If desired, the purge and aeration process 512 may include modifying the temperature of the sterilized object such as by using heated or cooled air during purge steps, and by heating or cooling the sterilization chamber walls. The purge and aeration process 512 may comprise rinsing the sterilization chamber with air following the dwell step while monitoring a residual gas in the chamber using a residual gas sensor until a residual gas concentration falls below a predetermined safety threshold, as shown at block 514. For example, an CD sensor in the chamber or in a flow path associated with the chamber, such as in an exhaust flow path, may be used to monitor CD levels during aeration until such levels fall below a threshold. The purge and aeration process 514 may be repeated until the CD concentration falls below a predetermined threshold. The predetermined threshold concentration may be based on known safe concentration of CD and/or detectable levels. For example, the predetermined threshold concentration may be those listed by safety regulators. In some examples, the predetermined threshold concentration may be higher than a concentration listed by regulators as a safe level. For example, there may be means adjacent to the sterilization chamber to mitigate sterilant in the sterilization chamber. For example, in some cases, dilution in the air that surrounds the sterilization chamber may be sufficient to reduce a residual sterilant level to a safe level. Alternatively, an air curtain or air exchange in the room surrounding the sterilization chamber may be sufficient to render the sterilant escaping the sterilization chamber safe.

Once the CD concentration is below the predetermined threshold, the control and monitoring system 104 may issue a control command to the access control system 120 to unlock the door the sterilization chamber 102, as shown at block 514. The door may not be unlocked or open until the CD concentration is below the predetermined threshold. For example, the user may not bypass the access control system 120 to open the sterilization chamber 102 while CD concentrations are above the predetermined threshold.

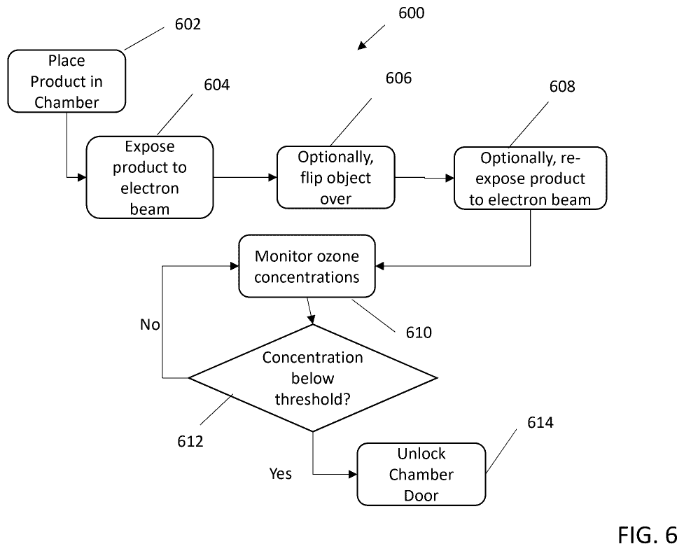

FIG. 6 is an illustrative flow chart of a method 600 for sterilizing an object using the sterilization apparatus 100 described herein and electron beam (e-beam) sterilization. To begin, an object to be sterilized may be placed in the sterilization cavity of the sterilizing chamber 102, as shown at block 602. In some cases, the sterilization cavity may be a large room with a conveyer belt to move the product through the room. Once the object has been placed in the sterilization cavity, the door of the sterilizing chamber 102 may be closed and locked. The door may be manually locked by a user or may be automatically locked using, for example, the access control system 120. For example, the access control system 120 may include an electronically actuated locking system which locks the door of the sterilizing chamber 102 in response to a command received from the control and monitoring system 104. The command may be issued in response to the closing of the door and/or the initiation of the sterilization procedure

The product may then be exposed to an electron beam, as shown at block 604. In one illustrative example, the product may travel on a conveyor belt which passes under an electron beam accelerator. The electron beam may shower electrons over the product. In some cases, the product may be flipped over, as shown at block 606. The product may then be once again exposed to the electron beam, as shown at block 608. This may allow the product to be sterilized from more than one direction to allow for relatively uniform sterilization.

As the free electrons are exposed to oxygen (O2) in the ambient air of the chamber 102, ozone (O3) may be created as a by-product of the e-beam sterilization process. It is contemplated that ozone may be a by-product of other radiation sterilization processes, including, but not necessarily limited to, gamma radiation, x-ray, and/or ultraviolet (UV) light. While the specific method steps for using these different types of radiation processes are not discussed, it is contemplated that each of the radiation sterilization processes may benefit from monitoring a by-product, such as ozone, of the sterilization apparatus 100 as described herein. Ozone may cause a variety of health problems in humans, including, but not limited to, headaches, coughing, dry throat, shortness of breath, chest pain, congestion, worsening of bronchitis, emphysema, and asthma, reduced lung function, lung lining inflammation, permanent lung tissue scarring, difficulty breathing deeply and vigorously, and/or the like.

Thus, it may be desirable to monitor enclosures that surround or are a part of a radiation sterilization system for ozone. An ozone detector may be positioned internal to the sterilization chamber 102 or external to the sterilization chamber 102 depending on the system set up to monitor ozone concentrations, as shown at block 612. Ozone may also be monitored in an exhaust duct, if desired, so that the detector can monitor concentration in gasses exiting the chamber, as a proxy for monitoring in-chamber concentrations, without being subjected to the gamma radiation. It is contemplated that ozone may be monitored during the sterilization process and/or after the sterilization process is complete. For example, when an enclosure surrounding the radiation sterilization system is accessible to a user during the sterilization process, the ozone detector may be continuously monitoring and/or measuring ozone within the enclosure. While not explicitly shown, in some examples, the sterilization chamber 102 and/or an enclosure thereof may be purged, aerated, and/or vented to remove ozone from the sterilization chamber 102 and/or the enclosure thereof.

If the ozone concentrations are above a predetermined threshold, the control and monitoring system 104 may issue a command to the access control system 120 to maintain the door of the sterilization chamber 102 and/or an access point to an enclosure surrounding the sterilization chamber 102 in a locked configuration. For example, if the ozone concentrations are at or above 0.10 ppm in the sterilization chamber 102 and/or the enclosure thereof, the control and monitoring system 104 may send a command to the access control system 120 to maintain the door of the sterilization chamber 102 and/or the access point to the enclosure in a locked configuration. When the ozone concentrations are below 0.10 ppm in the sterilization chamber 102 and/or the enclosure thereof, the control and monitoring system 104 may send a command to the access control system 120 to unlock the door of the sterilization chamber 102 and/or unlock the access point to the enclosure thereof, as shown at block 612. The door may not be unlocked or open until the ozone concentration is below the predetermined threshold. For example, the user may not bypass the access control system 120 to open the sterilization chamber 102 and/or enclosure while ozone concentrations are above the predetermined threshold.

In some embodiments, the predetermined threshold concentration may be those listed by safety regulators. In some examples, the predetermined threshold concentration may be higher than a concentration listed by regulators as a safe level. For example, there may be means adjacent to the sterilization chamber to mitigate sterilant in the sterilization chamber. For example, in some cases, dilution in the air that surrounds the sterilization chamber may be sufficient to reduce a residual sterilant level to a safe level. Alternatively, an air curtain or air exchange in the room surrounding the sterilization chamber may be sufficient to render the sterilant escaping the sterilization chamber safe.

In some of the preceding examples, the concentration of sterilant or sterilization byproduct is monitored following completion of at least some part of the sterilization procedure. The monitored concentration may be of gasses internal to the chamber, such as by having a sensor in the chamber itself, or by having a sensor in a duct that is operatively linked to the chamber, such as a duct that contains a circulation fan/blower, or an exhaust duct used to remove air/gasses from the chamber. The monitored concentration may be compared to one or more thresholds. One threshold may be a safety threshold used to determine when it is safe to open the sterilization chamber. Other thresholds may be used for process monitored, for example, a first purge/aeration step may be performed while monitoring concentration of the sterilant itself, when applicable, to confirm that in-process concentrations met intended process parameters. In another example, a first purge or aeration step may be performed while monitoring a concentration of additional substances, such as humidity or inert gasses, used during sterilization to confirm process parameters are met. In another example, a first purge or aeration step may be performed while monitoring a concentration of sterilization byproducts to confirm process parameters are met. As aeration and purge cycles continue, any of these concentrations may be monitored for changes from one step to another, as additional process monitors.

Once a safety threshold is met, the system may respond by removing a software-enforced lock on the system. This may or may not result in the sterilization chamber itself being opened. For example, a manual input may be required in addition to the software-enforced lock being released before the chamber is opened. An in-line process may be facilitated by automatically opening the chamber and allowing sterilized product to be removed. In still other examples, the chamber may be automatically opened while product remains in place.

The safety threshold may be an actual safety threshold, for example, referring to the previously described regulations on ETO concentration, an in-chamber concentration below 13 ppb may be the safety threshold. In other examples, the sterilization chamber may be positioned in a relatively larger room, and the safety threshold can be calibrated to the relative room sizes. Such calibration may account for typical and/or ambient ETO concentrations, which may be in the low single digit ppb. Thus, for example, assuming ambient ETO concentration of 2 ppb, a room size of 12,000 L (inclusive of the chamber), a chamber having a volume of 4,000 L, a safety threshold of 30 ppb for the in-chamber concentration may be sufficient (release would increase the room concentration of ETO to 10+2 ppb, below a 13 ppb threshold). Such thresholding may be further modeled and refined, however, the point is that the in-chamber concentration may be compared to a threshold that is calibrated to the surroundings of the sterilization chamber.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A method of sterilizing a medical device, comprising:
placing the medical device in a sterilization chamber;
locking a door of the sterilization chamber;
performing one or more steps of a sterilization procedure, the one or more steps comprising:
exposing the medical device to a sterilant; and
monitoring a concentration of the sterilant and/or a by-product of the sterilization procedure; and
after the sterilization procedure is complete, unlocking the door of the sterilization chamber in response to fulfilling both a) and b):
a) the monitored concentration of the sterilant and/or the by-product of the sterilization procedure being below a predetermined threshold; and
b) a request to unlock the chamber is received.

2. The method of claim 1, wherein the sterilant is nitrogen dioxide.

3. The method of claim 2, wherein the predetermined threshold is 10 parts per million of nitrogen dioxide.

4. The method of claim 2, wherein the predetermined threshold is 1 part per million of nitrogen dioxide.

5. The method of claim 2, wherein the by-product is nitrogen oxide.

6. The method of claim 5, wherein the predetermined threshold is a change in slope of a plot of a log of a concentration of the nitrogen oxide versus time.

7. The method of claim 1, wherein the sterilant is ethylene oxide.

8. The method of claim 7, wherein the predetermined threshold is 18 parts per billion of ethylene oxide.

9. The method of claim 7, wherein the predetermined threshold is 13 parts per billion of ethylene oxide.

10. The method of claim 1, wherein the sterilant is radiation.

11. The method of claim 10, wherein the by-product is ozone.

12. The method of claim 11, wherein the predetermined threshold is 0.10 parts per million of ozone.

13. A method of sterilizing a medical device, comprising:
placing the medical device in a sterilization chamber;
receiving at an access control system a command to lock a door of a sterilization system;
locking a door of the sterilization system;
performing one or more steps of a sterilization procedure, the one or more steps comprising:
exposing the medical device to a sterilant; and
monitoring a concentration of the sterilant and/or a by-product of the sterilization procedure;
in response to the concentration of the sterilant and/or the by-product of the sterilization procedure is below a predetermined threshold, enabling access to the sterilization chamber;
receiving at an access control system a request to unlock a door of the sterilization chamber; and either:
if access has been enabled, unlocking the door; or
if access has not been enabled, denying the request to unlock the door.

14. A sterilization apparatus, comprising:
a sterilization chamber having a lockable door;
at least one chemical sensor positioned within a chamber of the sterilization chamber;
an access control system in electronic communication with the lockable door;
a control and monitoring system in electronic communication with the at least one chemical sensor and the access control system; and
a sterilant source in fluid communication with the chamber of the sterilization chamber, the sterilant source providing a sterilant to the chamber of the sterilization chamber;
wherein the access control system is configured to lock the lockable door at a start of a sterilization procedure and unlock the lockable door as follows:
monitoring a concentration of the sterilant or a by-product of the sterilization procedure after the sterilant has been introduced to the chamber;
determining the concentration of the sterilant or by-product is below a predetermined threshold and, in response, enabling access to the chamber;
receiving a request to unlock a door of the sterilization chamber; and either:
if access has been enabled, unlocking the door; or
if access has not been enabled, denying the request to unlock the door.

15. The sterilization apparatus of claim 14, further comprising an enclosure surrounding the sterilization chamber.

16. The sterilization apparatus of claim 15, further comprising at least one chemical sensor positioned within the enclosure.

17. The sterilization apparatus of claim 14, further comprising one or more leak sensors external to the sterilization chamber.

18. The sterilization apparatus of claim 14, wherein the sterilant is nitrogen dioxide.

19. The sterilization apparatus of claim 14, wherein the sterilant is ethylene oxide.

20. The sterilization apparatus of claim 14, wherein the sterilant is an electron beam.
